(19) United States
(12) Patent Application Publication
(10) Pub. No.: US 2021/0349032 A1
(43) Pub. Date: Nov. 11, 2021

(54) IMAGE DETECTION FOR TEST STICK DIAGNOSTIC DEVICE RESULT CONFIRMATION

(71) Applicant: Church & Dwight Co., Inc., Princeton, NJ (US)

(72) Inventors: Albert Nazareth, Mercerville, NJ (US); Timothy Snowden, Howell, NJ (US); Sujata Sundara Rajan, Belle Mead, NJ (US)

(73) Assignee: Church & Dwight Co., Inc., Princeton, NJ (US)

(21) Appl. No.: 17/308,171

(22) Filed: May 5, 2021

Related U.S. Application Data
(60) Provisional application No. 63/020,764, filed on May 6, 2020.

(51) Int. Cl. G01N 21/77; G06T 7/73; G06K 9/62; G06F 3/14

(52) U.S. Cl. CPC G01N 21/77; G06T 7/73; B01L 3/5023; G06F 3/14; G06T 2207/30204; G06K 9/6267

(57) ABSTRACT

Machine image detection involving a trained classifier is used to detect a result of a test stick diagnostic device configured to detect the presence of an analyte in a test sample. Example implementations include receiving a digital image comprising a depiction of at least a portion of a test stick diagnostic device and applying the digital image to a classifier configured to determine a relative position, relative orientation, and relative scale of the portion of the test stick diagnostic device with respect to the digital image, identify a test result region of the test stick diagnostic device, and detect a test result marking in the test result region of the test stick diagnostic device. An indication of a result of a diagnostic test may be provided based on the detected test result marking.

### CROSS-REFERENCE TO RELATED APPLICATIONS

The present application claims priority to U.S. Provisional Patent Application No. 63/020,764, filed May 6, 2020, the disclosure of which is incorporated herein by reference in its entirety.

### TECHNOLOGICAL FIELD

The present disclosure relates generally to machine-based image detection and, in particular, to the use of such image detection within an artificial intelligence environment to recognize result indicators presented on diagnostic test kits, such as those used in connection with testing of pregnancy, ovulation, and semi-quantitative estimates of pregnancy progression.

### BACKGROUND

Home-based test kits designed to assist individuals in identifying any of a number of medical statuses or conditions have become increasingly popular among many users. For example, there are various test kits available on the market for detection of pregnancy at home. Such home-based pregnancy test kits are popular among consumers due to the convenience of use and reduced cost in comparison to visiting a doctor. In general, home-based pregnancy test kits detect pregnancy by detecting the presence of Human Chorionic Gonadotropin (hCG) hormone in urine. In many such test kits, the hCG test involves immersing the absorbent tip portion of the test stick in urine and monitoring a visual color change or appearance of two lines. Typically, the appearance of a color change or appearance of two lines (one control line and the other a test line) on the result window of the test stick confirms pregnancy.

However, the visual inspection for a color change or appearance of a test line on the result window can sometimes be confusing to a user, which can result in the user incorrectly interpreting the test results. The likelihood of user confusion or of the user incorrectly interpreting the test results often occurs in situations where the test results take the form of a very faint test line on the test stick. Such faint test lines are often incorrectly interpreted as a positive pregnancy test or a negative pregnancy test. As such, a user-based visual inspection is often insufficient to convey the diagnostic information to a user and often undermines the user's confidence in the results and the test device.

Therefore, it would be desirable to have a system and method that takes into account at least some of the issues discussed above, as well as other possible issues.

### BRIEF SUMMARY

Example implementations of the present disclosure are directed to the use of machine-based image detection within an artificial intelligence environment to recognize result indicators presented on diagnostic test kits, including but not limited to test kits and other test stick diagnostic devices used in connection with home-based testing for pregnancy, ovulation, and semi-quantitative estimates of pregnancy progression.

Some example implementations of the present disclosure overcome technical challenges associated with conventional approaches to test result interpretation that are y based on a user's visual perception of the test results by using image detection protocols to process a captured image of a test stick diagnostic device. Some such example implementations involve applying the image to a trained classifier configured to detect the relevant test result markings on the test stick diagnostic device and provide the user with an indication of the result. For example, the classifier may be trained using a significant training data set and assess the image at the pixel-level to detect faint or otherwise hard-to-see lines and accurately interpret test results that an individual user may be unable to definitively read.

It will be appreciated that some example implementations of the present disclosure involve a mobile application installed on a user's smartphone or other mobile device. In some such example implementations, the mobile application may leverage the mobile device's camera, processing capabilities, communication capabilities, and user interface to allow a user to use aspects of the present disclosure in a familiar technical environment.

Regardless of the precise form that an example implementation of the present disclosure may take or the context in which an example implementation is deployed, it will be appreciated that some example implementations of the present disclosure involve receiving a digital image depicting at least a portion of a test stick diagnostic device, applying the image to a classifier, and providing an indication of a result of a relevant diagnostic test based on test result markings detected by the classifier.

The present disclosure thus includes, without limitation, the following example implementations.

Some example implementations provide a method of detecting a result of a test stick diagnostic device, the method comprising receiving a digital image comprising a depiction of at least a portion of a test stick diagnostic device; applying the digital image to a classifier configured to determine a relative position, relative orientation, and relative scale of the portion of the test stick diagnostic device with respect to the digital image; identify a test result region of the test stick diagnostic device; and detect a test result marking in the test result region of the test stick diagnostic device; and providing an indication of a result of a diagnostic test based on the test result marking.

In some example implementations of the method of any preceding example implementation, or any combination of any preceding example implementations, the classifier being configured to determine the relative position, relative orientation, and relative scale of the portion of the test stick diagnostic device with respect to the digital image comprises the classifier being configured to detect a position, orientation, and scale of a set of markings on a surface of the test stick diagnostic device.

In some example implementations of the method of any preceding example implementation, or any combination of any preceding example implementations, the test result region of the test stick diagnostic device is an elliptical region on a surface of the test stick diagnostic device.

In some example implementations of the method of any preceding example implementation, or any combination of any preceding example implementations, the test result marking is a line disposed at a predetermined location within the test result region of the test stick diagnostic device.

In some example implementations of the method of any preceding example implementation, or any combination of any preceding example implementations, receiving the digital image comprises capturing the digital image via a camera.

In some example implementations of the method of any preceding example implementation, or any combination of any preceding example implementations, providing an indication of a result of a diagnostic test based on the test result marking comprises displaying a diagnostic result message on a user interface.

In some example implementations of the method of any preceding example implementation, or any combination of any preceding example implementations, the test stick diagnostic device is an immunological test stick diagnostic device configured to test a pregnancy status of a user.

Some example implementations provide an apparatus for detecting a result of a test stick diagnostic device, the apparatus comprising a memory configured to store computer-readable program code; and processing circuitry configured to access the memory, and execute the computer-readable program code to cause the apparatus to at least perform the method of any preceding example implementation, or any combination of any preceding example implementations.

Some example implementations provide a computer-readable storage medium for detecting a result of a test stick diagnostic device, the computer-readable storage medium being non-transitory and having computer-readable program code stored therein that, in response to execution by processing circuitry, causes an apparatus to at least perform the method of any preceding example implementation, or any combination of any preceding example implementations.

Some example implementations provide a method of detecting a result of a test stick diagnostic device, the method comprising receiving a digital image comprising a depiction of at least a portion of a test stick diagnostic device; providing the digital image to a classifier; receiving, from the classifier, an indication of a test result marking; and providing an indication of a result of a diagnostic test based on the test result marking; wherein the classifier is configured to determine a relative position, relative orientation, and relative scale of the portion of the test stick diagnostic device with respect to the digital image; identify a test result region of the test stick diagnostic device; and detect the test result marking in the test result region of the test stick diagnostic device.

In some example implementations of the method of any preceding example implementation, or any combination of any preceding example implementations, the classifier being configured to determine the relative position, relative orientation, and relative scale of the portion of the test stick diagnostic device with respect to the digital image comprises the classifier being configured to detect a position, orientation, and scale of a set of markings on a surface of the test stick diagnostic device.

In some example implementations of the method of any preceding example implementation, or any combination of any preceding example implementations, the test result marking is a line disposed at a predetermined location within the test result region of the test stick diagnostic device.

In some example implementations of the method of any preceding example implementation, or any combination of any preceding example implementations, the test stick diagnostic device is an immunological test stick diagnostic device configured to test a pregnancy status of a user.

Some example implementations provide an apparatus for detecting a result of a test stick diagnostic device, the apparatus comprising a memory configured to store computer-readable program code; and processing circuitry configured to access the memory, and execute the computer-readable program code to cause the apparatus to at least perform the method of any preceding example implementation, or any combination of any preceding example implementations.

Some example implementations provide a computer-readable storage medium for detecting a result of a test stick diagnostic device, the computer-readable storage medium being non-transitory and having computer-readable program code stored therein that, in response to execution by processing circuitry, causes an apparatus to at least perform the method of any preceding example implementation, or any combination of any preceding example implementations.

These and other features, aspects, and advantages of the present disclosure will be apparent from a reading of the following detailed description together with the accompanying figures, which are briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable unless the context of the disclosure clearly dictates otherwise.

It will therefore be appreciated that this Brief Summary is provided merely for purposes of summarizing some example implementations so as to provide a basic understanding of some aspects of the disclosure. Accordingly, it will be appreciated that the above described example implementations are merely examples and should not be construed to narrow the scope or spirit of the disclosure in any way. Other example implementations, aspects and advantages will become apparent from the following detailed description taken in conjunction with the accompanying figures which illustrate, by way of example, the principles of some described example implementations.

### BRIEF DESCRIPTION OF THE FIGURE(S)

Having thus described example implementations of the disclosure in general terms, reference will now be made to the accompanying figures, which are not necessarily drawn to scale, and wherein:

FIG. 1 illustrates an example test stick diagnostic device that may be used in connection with example implementations of the present disclosure

FIG. 2 illustrates an example image of a test result region and test result markings on an example test stick diagnostic device that may be used in connection with example implementations of the present disclosure.

FIG. 3 illustrates a system for detecting a result of a test stick diagnostic device, according to example implementations of the present disclosure;

FIG. 4A is a flowchart illustrating various steps in a method of detecting a result of a test stick diagnostic device, according to example implementations;

FIG. 4B is another flowchart illustrating various steps in a method of detecting a result of a test stick diagnostic device, according to example implementations;

FIG. 5 is an annotated example of an image that may be used in connection with determining the location, orientation, and scale of a test stick diagnostic device, according to example implementations;

FIG. 6 is a flowchart illustrating a decision flow that may be used by a classifier according to example implementations; and

FIG. 7 illustrates an apparatus according to some example implementations.

### DETAILED DESCRIPTION

Some implementations of the present disclosure will now be described more fully hereinafter with reference to the accompanying figures, in which some, but not all implementations of the disclosure are shown. Indeed, various implementations of the disclosure may be embodied in many different forms and should not be construed as limited to the implementations set forth herein; rather, these example implementations are provided so that this disclosure will be thorough and complete, and will fully convey the scope of the disclosure to those skilled in the art. For example, unless otherwise indicated, reference something as being a first, second or the like should not be construed to imply a particular order. Also, something may be described as being above something else (unless otherwise indicated) may instead be below, and vice versa; and similarly, something described as being to the left of something else may instead be to the right, and vice versa. Like reference numerals refer to like elements throughout.

Example implementations of the present disclosure are generally directed to improving the accuracy of a user's interpretation of test results provided by a test stick diagnostic device and the confidence users have in the test results provided by such test stick diagnostic devices. In particular, some example implementations of the present disclosure use machine-based image detection within an artificial intelligence environment to recognize result indicators presented on diagnostic test kits, including but not limited to test kits and other test stick diagnostic devices used in connection with home-based testing for pregnancy, ovulation, and semi-quantitative estimates of pregnancy progression.

As discussed herein, home-based pregnancy test kits have become popular among consumers due at least in part to the convenience of use and reduced cost in comparison to visiting a doctor. Some popular home-based pregnancy test kits detect pregnancy by detecting the presence of Human Chorionic Gonadotropin (hCG) hormone in urine. Many such tests involve the use of a test stick diagnostic device where a user immerses an absorbent tip portion or area of the test stick diagnostic device in urine and monitors a visual color change or appearance of two lines. Typically, the appearance of a color change or appearance of two lines (one control line and other test line) on the result window of the test stick diagnostic device confirms pregnancy.

Technical challenges can arise in the use of such test stick diagnostic devices when the user's visual inspection of a color change is inconclusive to the user such as when the appearance of a test line is faint, or when the user misinterprets the meaning of one or more lines that may appear. Since the appearance of the test line is governed at least in part by the underlying chemical reactions that occur within the test stick diagnostic device, there is often no way for the test stick diagnostic device to enhance the appearance of the test line or other indicator without impacting the accuracy and reliability of the underlying test chemistry. These technical challenges may be compounded when a user is relatively inexperienced with the use of a given test stick diagnostic device, lacks confidence in their visual acuity, or otherwise lacks experience in discerning a positive test result from a negative test result.

To address these, and other technical challenges, example implementations of the present disclosure apply a received digital image of at least a portion of a test stick diagnostic device to a classifier configured to detect a test result marking in a test result region of the test stick diagnostic device and provide an indication of the result of the relevant diagnostic test to a user based on the detected test result marking.

Many example implementations of the present disclosure arise in the context of test stick diagnostic devices used to test a user's pregnancy and/or ovulation status. As such, many of the examples described or otherwise disclosed herein use images, terms, and concepts that may be particular to such contexts. However, it will be appreciated that the use of images, terms, and concepts that may be associated with tests of a user's pregnancy or ovulation status in connection with certain examples should not be interpreted as limiting example implementations of the present disclosure to such contexts. Rather, some example implementations of the present disclosure may be suitable for use in various other contexts involving other diagnostic devices.

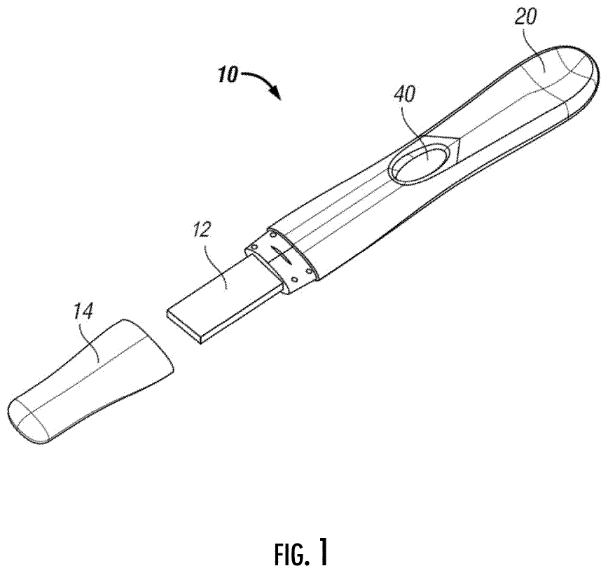

As noted herein, many example implementations of the present disclosure involve a test stick diagnostic device. FIG. 1 is an illustration of an example test stick diagnostic device 10 that may be used in connection with example implementations of the present disclosure. The test stick diagnostic device 10 shown in FIG. 1 is an example test stick diagnostic device. In some example implementations of the present disclosure, a test stick diagnostic device is an over-the-counter (OTC) or point of care (POC) test device for detecting an analyte in a sample. The test stick diagnostic device may include a proximal portion (e.g., a sample receiving member) in fluid communication with a distal portion (e.g., a reservoir). The proximal and distal portions may be interconnected by a substrate material, which itself may form all or part of the proximal and/or distal portion of the device. A liquid sample (e.g., urine) can be directly or indirectly deposited on the proximal portion of the device for transport to the distal portion. In some example implementations, the liquid sample flows across the substrate so as to contact one or more antibodies attached to or otherwise deposited on the substrate. The antibodies can be designed and/or chosen to recognize a variety of analytes. In some example embodiments, a test stick diagnostic device can encompass detection of glycoproteins, such as human chorionic gonadotropin (hCG), luteinizing hormone (LH), and follicle stimulating hormone (FSH).

It will be appreciated that test stick diagnostic devices that may be used in connection with example implementations described or otherwise disclosed herein can use a variety of techniques for detecting the presence of an analyte. One example is a sandwich technique wherein one or more antibodies used in the detection comprise a binding member or site which binds to an epitope on the analyte for detection. A labeled antibody reacts with the analyte to form a complex in the liquid sample. The analyte, which is bound with the labeled antibody or antibodies, reacts with one or more capture antibodies to form a “sandwich,” comprising the capture antibody, analyte (or antigen), and the labeled antibody. Each sandwich complex thus produced comprises three components: one capture antibody, one antigen, and one labeled antibody. An antibody used in such example implementations can be a polypeptide substantially encoded by an immunoglobulin gene or immunoglobulin genes, or fragments thereof, which may specifically recognize and bind an antigen. The recognized immunoglobulin genes include the kappa, lambda, alpha, gamma, delta, epsilon, and mu constant region genes, as well as the immunoglobulin variable region genes. Antibodies include fragments, such as Fab′, F(ab)₂, Fabc, and Fv fragments. The term antibody also can include antibody fragments either produced by the modification of whole antibodies or those synthesized de novo using recombinant DNA methodologies, and further can include “humanized” antibodies made by conventional techniques. Although polyclonal antibodies can be used, antibodies are preferably monoclonal antibodies. A capture antibody according to the disclosure can be an antibody attached to a substrate directly or indirectly, such as a solid substrate. The capture antibody can include at least one binding member that specifically or preferentially binds a particular distinct epitope of an antigen.

In example test stick diagnostic devices using the sandwich technique, the makeup of each sandwich complex can vary depending upon the particular labeled antibody (and thus the particular antigen) included therein. In the same test, there can be multiple different types of sandwiches produced. The sandwich complexes are progressively produced as the test liquid with the analyte therein continuously moves along the substrate of the device. As more and more of the analyte and/or labeled antibody complex is immobilized in sandwich form with the capture antibody or antibodies at the capture site, the label components aggregate and become detectable in that the accumulation of the sandwich complexes at the capture site can be detected in various ways, such as by visual inspection of, for example, color development at the capture site. Although the sandwich technique is provided as an exemplary embodiment, the test stick diagnostic devices that may be used in connection with example implementations described herein with respect to the improved detection and interpretation of test results provided by such test stick diagnostic devices are not limited to such an underlying technique. Rather, other techniques for identifying an analyte in a test sample and forming a detectable image or other indication based on the presence or absence of the analyte in the sample can be used.

Some example techniques for forming a detectable indication of a test result involve the use of a conjugate comprising one or more antibodies bound to detectable label components (e.g., colored particles, such as a metal sol or colloid particles). One or more of the antibodies used in some example test stick diagnostic devices (e.g., one or two) can be labeled. Any detectable label recognized in the art as being useful in various assays can be used. In particular, the detectable label component can include compositions detectable by reflective, spectroscopic, photochemical, biochemical, immunochemical, or chemical means. As such, the label component produces a detectable indication. For instance, suitable labels include soluble dyes, fluorescent dyes, chemiluminescent compounds, radioisotopes, electron-dense reagents, enzymes, colored particles, or dioxigenin. The label component can generate a measurable signal, such as radioactivity, fluorescent light, color, or enzyme activity, which can be used to identify and quantify the amount of label bound to a capture site. Thus, the label component can also represent the presence or absence of a particular antigen bound thereto, as well as a relative amount of the antigen (e.g., relative to a known standard, threshold standard, or a different standard).

Some example test stick diagnostic devices that may be used in connection with example implementations of the present disclosure can involve a biphasic chromatographic medium (substrate/test strip) and can involve an upstream release medium joined to a downstream capture medium. The release and capture media can comprise two different materials or phases having different specific characteristics. The two phases can be joined together to form a single liquid path such that a solvent front can travel unimpeded from the proximal (upstream) end of the release medium (which can be defined as a proximal portion of the biphasic medium) to the distal (downstream) end of the capture medium (which can be defined as a distal portion of the biphasic medium). A sample receiving member can be generally provided at the proximal end of the biphasic substrate and a reservoir of sorbent material can be located beyond the biphasic substrate. In certain embodiments, use of a biphasic chromatographic medium may enhance the speed and sensitivity of an assay, such as those described in U.S. Pat. Nos. 6,319,676; 6,767,714; and 7,045,342, which are incorporated herein by reference, including without limitation for the purpose of describing biphasic chromatographic media. Methods for manufacturing biphasic chromatographic media are also described in detail in U.S. Pat. No. 5,846,835, the disclosure of which is incorporated herein by reference in its entirety.

Reagents for detecting, labeling, and capturing an analyte of interest can be disposed on the release and capture media. In certain embodiments, one or more labeled conjugates can be located on the release medium and each can include a binding member (e.g., antibody) that may be reactive with a particular site (sometimes referred to as a “first epitope,” “second epitope,” etc.) on the analyte of interest. The labeled conjugates further can comprise one or more detectable markers (or labels), as discussed herein.

The release medium can be formed from a substance which allows for release of reagents deposited thereon, which can comprise reagents that are releasably (i.e., not permanently) bound to the release medium. The primary function of the release medium is first to support and to subsequently release and transport various immunological components of the assay, such as a labeled conjugate and/or a capturable conjugate, both of which are capable of binding to the analyte of interest. The release medium can be formed of any material capable holding, releasing, and transporting various immunological parts of the test such as the labeled test component (e.g., a bibulous, hydrophilic material).

The capture medium can be formed from a material which permits immobilization of reagents for detection of the presence of analyte in the test fluid. Immobilization can refer to any interaction that results in antibodies or analytes being irreversibly bound to the substrate such that they are not appreciably washed away, e.g., during the course of a single use of the device. The capture medium can comprise hydrophilic polymeric materials, such as microporous films or membranes, which permit protein reagents to be immobilized directly on the membrane by passive adsorption without the need for chemical or physical fixation, although fixation such is not excluded.

The release medium and capture medium can be joined via any suitable means. For example, the two media can be joined by overlapping the downstream edge of the release medium over the upstream edge of the capture medium, then adhering the resulting biphasic material to a clear polymer film or opaque sheet, thereby holding the media in place. Alternately, the media can be connected by a non-overlapping butt joint.

The diffusible and non-diffusible reagents can be applied to the release and capture media, respectively, by any suitable technique. In one embodiment, the diffusible antibody reagents can be applied to the release medium by direct application onto the surface of the medium and dried to form a band. Generally, reagents can be immobilized using absorption, adsorption, or ionic or covalent coupling, in accordance with any suitable methods.

Regardless of the techniques and technology used to identify an analyte in a test sample, example implementations of a test stick diagnostic device (such as a test stick diagnostic device used as part of a home pregnancy or ovulation status test, for example), may take the form of the test stick diagnostic device 10 shown in FIG. 1. The casing of test stick diagnostic device 10 is generally configured to provide a recessed portion 20 shaped to permit users to place their thumb into the recessed portion and their forefinger on the bottom of the casing to securely hold the test stick diagnostic device 10. Disposed within the casing are a sample receiving member 12, the biphasic chromatographic substrate(s), and reservoir absorbent. The sample receiving member 12 can be disposed within the casing, extend to the exterior thereof, and may be covered by a removable cap 14. A biphasic chromatographic substrate comprising a release medium and a capture medium joined together to form a single liquid path may be located downstream of the sample receiving member 12 within the casing of the test stick diagnostic device 10. The release and capture media can be laminated onto a transparent or opaque plastic film or sheet. By providing a reservoir of sorbent material (e.g., absorbent paper made form cotton long linter fibers or cellulosic materials) disposed beyond the chromatographic substrate, a relatively large volume of the test liquid and any analyte it contains can be drawn through the test area to facilitate background clearance and thereby aid sensitivity. The reservoir absorbent generally facilitates capillary action along the chromatographic substrate and absorbs excess liquid contained within the device.

In some example implementations, the relevant test sample passes through the biphasic chromatographic substrate and into reactive contact with the test site (e.g., the capture site), and optionally one or more control sites. A test result region 40 on the top of the casing defines a region that permits a user to observe test results as they become detectable. As described herein, “becoming detectable” specifically can relate to the development and display of a test result marking, such as one or more lines in a color that differs from the background of the test result region 40.

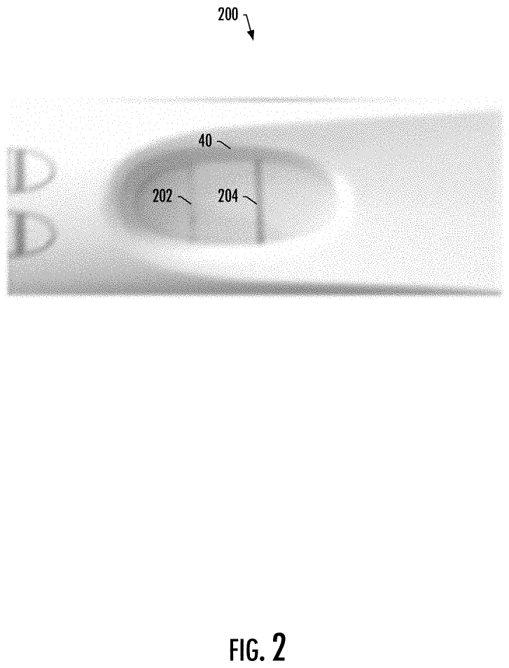

FIG. 2 illustrates an example image of a test result region 40 and test result markings 202 and 204 on an example test stick diagnostic device that may be used in connection with example implementations of the present disclosure. As shown in FIG. 2, the test result region 40 may, in some example implementations, be elliptically shaped and provide a view of two test result marking 202 and 204. While the test result region 40 is shown in FIG. 2 to be shaped in an ellipse, it will be appreciated that any shape may be used in other example implementations of a test result region 40.

In some example implementations of a test stick diagnostic device, test result marking 204 is used to indicate to a user that the test sample has been applied to the test stick diagnostic device, and test result marking 202 is used to indicate the presence of the relevant analyte in the test sample. As shown in FIG. 2, test result marking 202 may, in some situations, appear to be more faint, thinner, lower in color value, and/or less continuous than the test result marking 204. As such, in some situations, a user may incorrectly interpret the test results based on one or more faulty assumptions or understandings of the extent to which the test result marking 202 must appear in order to confirm a given test result. While the example presented in FIG. 2 involves test result markings that are in the form of a colored line, it will be appreciated that example implementations of the present disclosure are not limited to only detecting the presence or absence of one or more lines, and test result markings 202 and 204 may include any shape, symbol, or other visual indicator that can be captured in a digital image.

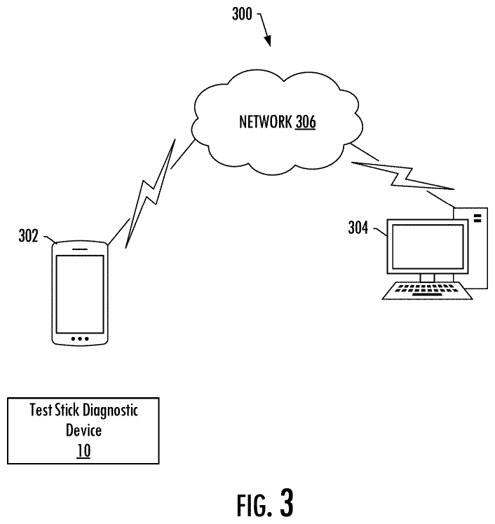

FIG. 3 illustrates a system for detecting a result of a test stick diagnostic device, according to example implementations of the present disclosure. The system may include any of a number of different subsystems (each an individual system) for performing one or more functions or operations. As shown in FIG. 3, the system 300 includes the test stick diagnostic device 10, a user device 302, and a classifier management system 304. In some example implementations of system 300, the test stick diagnostic device 10, user device 302, classifier management system 304, and any other subsystems or other elements that may be included in system 300 may be co-located or directly coupled to one another, or in some examples, various ones of the elements may communicate with one another across one or more computer networks 306. Further, although shown as part of the system 300, it should be understood that any one or more of the above may function or operate as a separate system without regard to any of the other subsystems. It should also be understood that the system 300 may include one or more additional or alternative subsystems than those shown in FIG. 3.

As shown in FIG. 3, some example implementations of system 300 involve the use of a user's smartphone as the user device 302. In some such example implementations, a mobile application and/or other software stored on the user device 302 is used to implement one or more aspects of the present disclosure. For example, in situations where the user device 302 is a smartphone equipped with a camera, the camera may be used to capture a digital image of the test stick diagnostic device 10. Once the image is captured, the image may be received by a mobile application or other software configured to implement one or more aspects of the present disclosure, and the received image may be applied to a classifier. In some example implementations, the classifier may be stored on or otherwise reside on the user device 302. In such situations, the user device 302 may communicate with the classifier management system 304 to receive updates to the classifier, to provide information to the classifier management system (such as images captured by the user device 302 and/or determinations made by the user device 302, for example), or otherwise exchange messages and/or other data regarding the classifier. In situations where the classifier is not stored or the user device 302, the user device 302 may transmit the image of the test stick diagnostic device 10 to the classifier management system 304, where the image may be processed by the classifier and an indication of the result of the relevant diagnostic test may be transmitted back to the user device 302.

Regardless of whether the classifier resides on the user device 302, the classifier management system 304, or on another system within or in communication with system 300, the classifier, upon receipt of the image of test stick diagnostic device 10 may begin processing the image.

In some example implementations, the classifier detects the relative position, relative orientation, and relative scale of the portion of the test stick diagnostic device 10 captured in the image. It will be appreciated that differences in cameras, the photography technique of the user, and the location of the test stick diagnostic device 10 with respect to the camera and the camera's field of view can result in images where the appearance of the test stick diagnostic device or portions thereof vary widely from image to image. For example, the position of test stick diagnostic device 10 may vary with relative to the horizontal and vertical axis of the image. In some example implementations, the test stick diagnostic device 10 may appear to be rotated or placed at an angle with respect to the edges of the image. In some example implementations, the distance between the test stick diagnostic device 10 and the lens of the camera may cause the test stick diagnostic device 10 or portions thereof to appear larger or smaller with respect to other elements in the image.

In some example implementations, the classifier determines the relative position, relative orientation, and relative scale of a portion of the test stick diagnostic device by detecting a feature on the casing of the test stick diagnostic device. In some example implementations, the feature may be one or more words printed in a known position and a known size on the test stick diagnostic device. For example, some test stick diagnostic devices used in connection with home-based pregnancy tests include the words “pregnant” and “not pregnant” in a known position and orientation on the side of the casing of the test stick diagnostic device that also contains the test result region of the test stick diagnostic device. In such example implementations, the known attributes of the printed words such as their position, orientation, and length, for example, can be compared by the classifier to the position, orientation, and length of the words detected in the image.

In some example implementations, the classifier uses the location, size, and angle of the printed words or other feature to identify a test result region on the test stick diagnostic device (such as test result region 40 on the test stick diagnostic device 10, for example). In some example implementations, the position of the test result region relative to the location of the feature is known, and can thus be calculated relative to the detected position, orientation and scale of the test stick diagnostic device.

Upon identifying the test result region, the classifier may then analyze the pixels in the test result region to detect the presence or absence of one or more test result markings in the test result region. Based on the presence or absence of one or more relevant test result markings, the classifier can then provide an indication of a result of a diagnostic test, which may be presented to a user, for example, via a user interface of the user device 302.

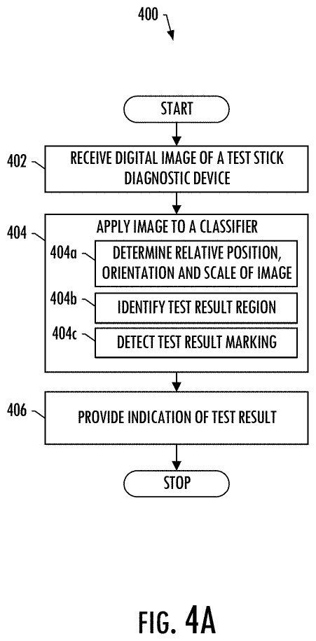

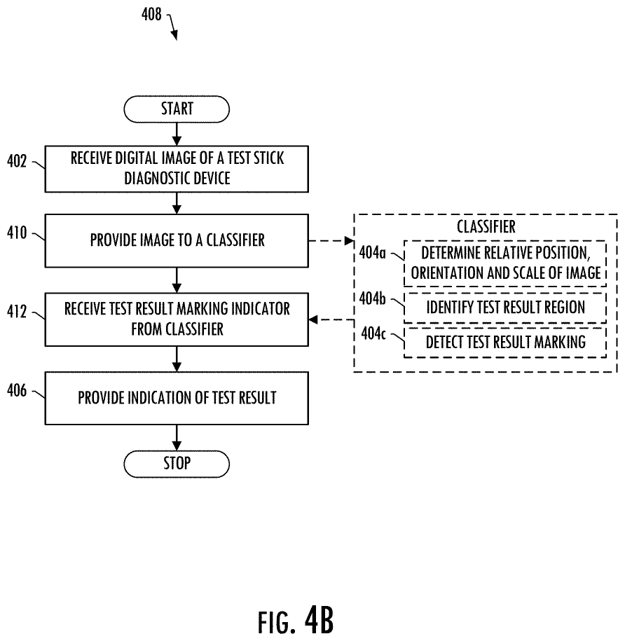

FIG. 4A is a flowchart illustrating various steps in a method 400 for detecting a result of a test stick diagnostic device, according to example implementations of the present disclosure. As shown at block 402, the method includes receiving a digital image of a test stick diagnostic device. Some example implementations of block 402 include receiving a digital image comprising a depiction of at least a portion of a test stick diagnostic device. It will be appreciated that any of the test stick diagnostic devices described or otherwise disclosed herein, including but not limited to those described in connection with test stick diagnostic device 10 in FIG. 1, may be used in connection with example implementations of block 402. For example, the test stick diagnostic device may be an immunological test stick diagnostic device configured to test a pregnancy status of a user. In some such example implementations, and in other example implementations of block 402, receiving the digital image comprises capturing the digital image via a camera. For example, a camera, such as a camera incorporated into user device 302, may be used to capture an image of all or part of a test stick diagnostic device.

As shown at block 404, the method 400 also includes applying the digital image to a classifier. As discussed and otherwise disclosed herein, some example implementations of block 404 involve a trained classifier model that has be trained using a data set including multiple images of test stick diagnostic devices with known results such that the classifier is able to analyze images (such as at a pixel level, for example) to detect features of the relevant test stick diagnostic device and one or more test result markings displayed on the test stick diagnostic device.

As shown at subblock 404a, the classifier is configured to determine a relative position, relative orientation, and relative scale of the portion of the test stick diagnostic device with respect to the digital image. In some situations, the position, orientation, and size or scale of the test stick diagnostic device may vary from image to image. As such, in some example implementations, the classifier first determines how image elements depicting all or part of a test stick diagnostic device appear in the image, and subsequently determines the position of other image elements (such as those associated with a test result region and one or more test result markings, for example), based on the relative position, orientation, and scale of the test stick diagnostic device as it appears in the image. In some example implementations of subblock 404a, the classifier being configured to determine the relative position, relative orientation, and relative scale of the portion of the test stick diagnostic device includes the classifier being configured to detect a position, orientation, and scale of a set of markings on a surface of the test stick.

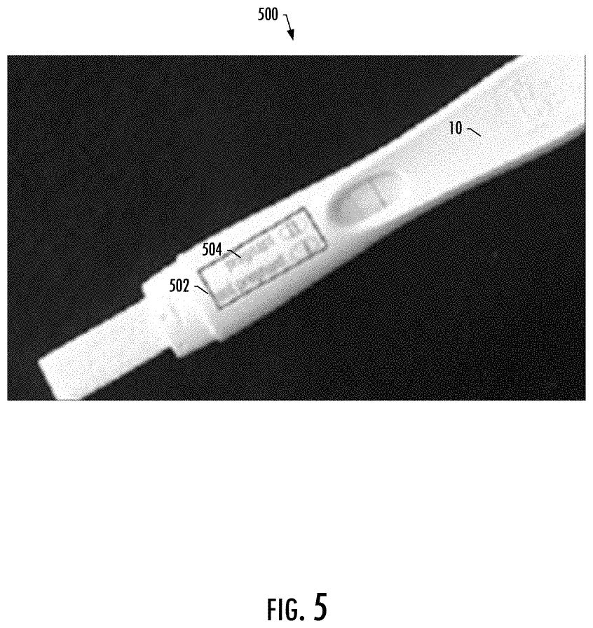

FIG. 5 is an annotated example of an image 500 that may be used in connection with determining the location, orientation, and scale of a test stick diagnostic device, according to example implementations. As shown in FIG. 5, an example test stick diagnostic device 10 is shown as having markings 504 (in the form the words in the example shown in FIG. 5) in a region 502 on the surface of the test stick diagnostic device 10. In some example implementations, in addition to using the detection of a predetermined set of markings on the surface of the relevant test stick diagnostic device to assist in determining how the test stick diagnostic device is positioned, oriented, and scaled in the image, the detection of the set of markings can also be used to quickly determine if an image is invalid in the sense that it cannot be processed by the classifier. For example, if the classifier is unable to detect the set of markings, the test stick diagnostic device may be flipped or otherwise positioned such that the test result region or its position cannot be viewed or confirmed. In some situations, a failure to detect an expected set of markings may indicate that the test stick diagnostic device is not one that the classifier is capable of assessing. Regardless of the cause of a failure to detect the markings (such as markings 504, for example), the failure to detect the markings may cause to classifier to provide an indication to the user (such as via the user interface of the user device 302, for example) that the image is invalid and cannot be processed.

In some example implementations of subblock 404a, the determination of the position, orientation, and scale of the markings (such as markings 504 in FIG. 5, for example) facilitate the determination of the scale and slant angle of the test stick diagnostic device depicted in the image. In situations where the markings include characters, such as in FIG. 5, for example, the position and appearance of the markings 504 in the relevant region 502 can be leveraged to rapidly determine the relevant position, orientation, and scale. As shown in FIG. 5, the characters in markings 504 in the region 502 is dark in color and the background of region 502 is white. As such, the contrast between the markings 504 and the background of region 502 can be used to detect the markings 504. Moreover, since the characters in markings 504 form text with a width is larger than its height, the slant angle can be detected accurately.

In some example implementations, of subblock 404a, several substeps are used to detect the position orientation, and scale of the relevant markings based on contrast between the markings and the background or adjacent portions of the test stick diagnostic device. In an example pyramid construction substep, four image pyramids are constructed based on the relevant region's size. For example, the sizes of the pyramids are may be set to 800, 500, 300, or 1200 pixels.

After the construction of the image pyramids, a candidate point detection process can be applied to the image by applying one or more thresholds to account for the characters or other markings being darker than their respective background regions. In some example implementations, the threshold value is decided using a mean value in a defined neighborhood of each point. In such example implementations, if the intensity value is smaller than 95% of the relevant mean value, the point is considered as candidate point and if not, it is considered as the background. After thresholding, morphology closing operation can be applied to make a connected blob of foreground points. In example implementations where the terms “pregnant” and “not pregnant” are used (such as in FIG. 5, where those terms appear as markings 504 in region 502), a candidate region detection protocol may be used to further determine the position and orientation of the markings. For example, since the markings 504 in region 502 of FIG. 5 form two, parallel, thick lines, the relevant points can be classified as blobs for the purpose of a blob analysis. In example implementations involving blob analysis, the candidate region blobs are detected based on predetermined information regarding the width and height of the blobs. In some such example implementations, the slant angle of one or more blobs can be determined by calculating the covariance matrix of points in a blob, calculating the Eigen vectors x₁, x₂ corresponding to eigen value λ₁, λ₂ (λ₁>λ₂) of the covariance matrix, and calculating the slant angle of the blob by the following operation α=−α tan 2(x₂₁, x₁₁).

In some example implementations, after the slant angle is calculated, four vertices of the relevant blob can be determined using the slant angle difference of two-character candidate region overlap and distance. For example, if the rotation angle difference of two-character candidate regions is less than 10 degrees, overlap in the horizontal direction is more than 40%, and the distance is less than 40 pixels, then the pair may be determined to be a candidate character region pair.

In example implementations where the slant angle of every relevant blob has been determined, final character region detection and image normalization may be performed using an angle rotation approach, and one or more of a binary gradient pattern, boosting algorithm, Support Vector Machine training algorithm, or the like.

As shown in FIG. 4, block 404 may also include subblock 404b, which includes identifying a test result region of the test stick diagnostic device. In some example implementations, such as those shown in connection with the test result region 40 in FIG. 2, for example, the test result region may be an elliptical region on a surface of the test stick diagnostic device.

Some example implementations of block 404b employ similar approaches to those used in example implementations of block 404a, and the classifier may be trained to identify a test result region based on the known shape of test result region. For example, pyramid images (for example, three pyramid images) may be constructed to determine the scale of the test result region the classifier may be used to process the pyramid images and apply a non-maximum value suppression and overlapping approach to identifying the test result region.

As shown in FIG. 4, some example implementations of block 404 include subblock 404c, which includes detecting a test result marking in the test result region of the test stick diagnostic device. Some example implementations of block 404c involve a test result marking that is a line disposed at a predetermined location within the test result region of the test stick diagnostic device. In some example implementations of block 404c, the detection of a test result marking in the test result region is based on the training performed on the classifier, which is used to assess the portions of the image contained within the test result region.

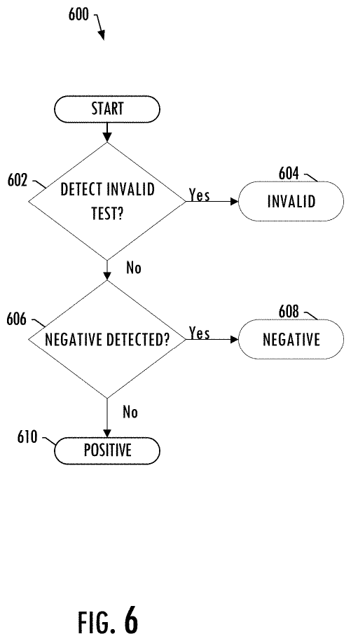

In example implementations of block 404c that involve a test stick diagnostic device that is configured to detect the presence or absence of an analyte, the classifier can be trained to account for three outcomes, as shown in FIG. 6. FIG. 6 is a flowchart illustrating a decision flow 600 that may be used by a classifier according to example implementations. As shown in FIG. 6, the decision flow first includes deciding, at block 602, whether the test was detected as invalid, such as by failing to identify one or more markings that would allow for the determination of the relative position, relative orientation, and relative scale of the test stick diagnostic device as shown in an image, or by failing to identify the test result region, for example. If the test was determined at block 602 to be invalid, the decision flow 600 transitions to block 604, where an indication that the test is invalid can be provided.

As shown at block 606, if the test was not determined at block 602 to be invalid, the classifier may then determine if a negative test result is detected. If a negative result is detected, the decision flow 600 may transition to block 608, and an indication of a negative test may be provided. If a negative test result was not detected, the process flow 600 may transition to block 610, and an indication of a positive result may be provided.

In some example implementations, of block 404c, the three classes of answers (invalid, negative, and positive) shown in FIG. 6 can be used to configure the classifier. For example, three pyramid images can be configured to solve for a scale change of the test result region, and the classifier can be applied to every pyramid image. Depending on the particular test result markings associated with a given test stick diagnostic device, the result of positive or negative (or pregnant or not pregnant, in examples involving a test stick diagnostic device configured to test a pregnancy status of a user, for example), can be made based on whether a threshold number of points are reached indicating a positive result.

As shown at block 406, the method 400 also includes providing an indication of the test result. Some example implementations of block 406 include providing an indication of a result of a diagnostic test based on the test result marking, such as the test result marking detected by the classifier. In some such example implementations, providing an indication of the result of the diagnostic test based on the test result marking includes displaying a diagnostic result message on a user interface. For example, the user interface of the user device 302 may be used to display a relevant message, such as “positive,” “negative,” “pregnant,” “not pregnant,” or the like.

FIG. 4B is a flowchart illustrating various steps in another method 408 for detecting a result of a test stick diagnostic device, according to example implementations of the present disclosure. As with the method 400 in FIG. 4A, the method 408 begins with block 402. As shown at block 402, the method includes receiving a digital image of a test stick diagnostic device. Some example implementations of block 402 include receiving a digital image comprising a depiction of at least a portion of a test stick diagnostic device. It will be appreciated that any example implementation of block 402 that may be used in instances of the method 400 may be used in instances of the method 408.

As shown at block 410, the method 408 includes providing the digital image to a classifier. Any approach to providing the digital image to the classifier may be used in example implementations of block 404. For example, the image may be applied directly to the classifier, as in some example implementations of block 404 of method 400. In other example implementations of block 410, one or more digital images may be transmitted (either singularly or in a batch or other grouping) to a remote classifier. In some such example implementations, the classifier management system 304 may direct images received from one or more user devices 302 to a classifier. The classifier may be local to or remote to the classifier management system, and the classifier management system and/or the user devices may use a network 306 or another network to pass the relevant digital image(s) to the classifier.

Upon receipt of one or more digital images, the classifier may process the images to detect a test marking or other relevant portion of the image(s). For example, the classifier may perform any combination of the steps, operations, and other processes described and otherwise disclosed herein, such as those presented herein with respect to blocks 404, 404a, 404b, 404c, FIG. 5, and FIG. 6. In some example implementations, the classifier is configured to at least determine a relative position, relative orientation, and relative scale of the portion of the test stick diagnostic device with respect to the digital image; identify a test result region of the test stick diagnostic device; and detect the test result marking in the test result region of the test stick diagnostic device.

As shown in block 412, the method 408 also includes receiving, from the classifier, an indication of a test result marking. As discussed and otherwise disclosed herein, the classifier is configured to detect a test result marking in a relevant region of an image, such as a line in a given position on a home pregnancy diagnosis test kit or other device. In some example implementations of block 412, the classifier management system 304 receives, from the classifier, an indication of the test result marking, which may be transmitted via one or more networks 306 from the classifier to the classifier management system. In some example embodiments of block 412, the classifier may pass the indication of the test result marking directly to one or more user devices 302, and the user device may receive the indication of the test result marking via a network 306.

As shown in FIG. 4B, the method 408 also includes block 406, which involves providing an indication of a result of a diagnostic test based on the test result marking. Any approach that may be used in example implementations of block 406 as described or otherwise disclosed herein, such as those described in connection with method 400, for example, may be used in example implementations of block 406 in the method 408.

According to example implementations of the present disclosure, the system 300 and its subsystems including the user device 302 and the classifier management system 304, for example may be implemented by various means. Means for implementing the system and its subsystems may include hardware, alone or under direction of one or more computer programs from a computer-readable storage medium. In some examples, one or more apparatuses may be configured to function as or otherwise implement the system and its subsystems shown and described herein. In examples involving more than one apparatus, the respective apparatuses may be connected to or otherwise in communication with one another in a number of different manners, such as directly or indirectly via a wired or wireless network or the like.

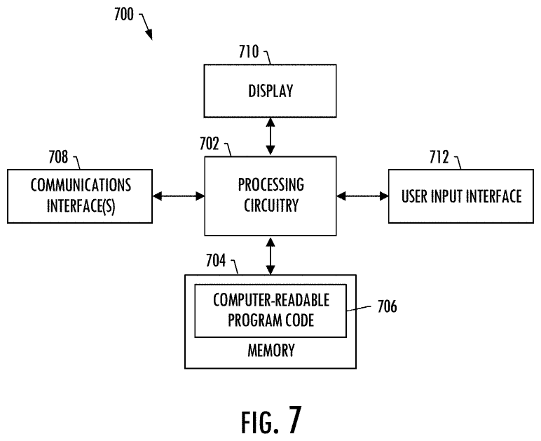

FIG. 7 illustrates an apparatus 700 according to some example implementations of the present disclosure. Generally, an apparatus of exemplary implementations of the present disclosure may comprise, include or be embodied in one or more fixed or portable electronic devices. Examples of suitable electronic devices include a smartphone, tablet computer, laptop computer, desktop computer, workstation computer, server computer or the like. The apparatus may include one or more of each of a number of components such as, for example, processing circuitry 702 (e.g., processor unit) connected to a memory 704 (e.g., storage device).

The processing circuitry 702 may be composed of one or more processors alone or in combination with one or more memories. The processing circuitry is generally any piece of computer hardware that is capable of processing information such as, for example, data, computer programs and/or other suitable electronic information. The processing circuitry is composed of a collection of electronic circuits some of which may be packaged as an integrated circuit or multiple interconnected integrated circuits (an integrated circuit at times more commonly referred to as a “chip”). The processing circuitry may be configured to execute computer programs, which may be stored onboard the processing circuitry or otherwise stored in the memory 704 (of the same or another apparatus).

The processing circuitry 702 may be a number of processors, a multi-core processor or some other type of processor, depending on the particular implementation. Further, the processing circuitry may be implemented using a number of heterogeneous processor systems in which a main processor is present with one or more secondary processors on a single chip. As another illustrative example, the processing circuitry may be a symmetric multi-processor system containing multiple processors of the same type. In yet another example, the processing circuitry may be embodied as or otherwise include one or more ASICs, FPGAs or the like. Thus, although the processing circuitry may be capable of executing a computer program to perform one or more functions, the processing circuitry of various examples may be capable of performing one or more functions without the aid of a computer program. In either instance, the processing circuitry may be appropriately programmed to perform functions or operations according to example implementations of the present disclosure.

The memory 704 is generally any piece of computer hardware that is capable of storing information such as, for example, data, computer programs (e.g., computer-readable program code 706) and/or other suitable information either on a temporary basis and/or a permanent basis. The memory may include volatile and/or non-volatile memory, and may be fixed or removable. Examples of suitable memory include random access memory (RAM), read-only memory (ROM), a hard drive, a flash memory, a thumb drive, a removable computer diskette, an optical disk, a magnetic tape or some combination of the above. Optical disks may include compact disk-read only memory (CD-ROM), compact disk-read/write (CD-R/W), DVD or the like. In various instances, the memory may be referred to as a computer-readable storage medium. The computer-readable storage medium is a non-transitory device capable of storing information, and is distinguishable from computer-readable transmission media such as electronic transitory signals capable of carrying information from one location to another. Computer-readable medium as described herein may generally refer to a computer-readable storage medium or computer-readable transmission medium.

In addition to the memory 704, the processing circuitry 702 may also be connected to one or more interfaces for displaying, transmitting and/or receiving information. The interfaces may include a communications interface 708 (e.g., communications unit) and/or one or more user interfaces. The communications interface may be configured to transmit and/or receive information, such as to and/or from other apparatus(es), network(s) or the like. The communications interface may be configured to transmit and/or receive information by physical (wired) and/or wireless communications links. Examples of suitable communication interfaces include a network interface controller (NIC), wireless NIC (WNIC) or the like.

The user interfaces may include a display 710 and/or one or more user input interfaces 712 (e.g., input/output unit). The display may be configured to present or otherwise display information to a user, suitable examples of which include a liquid crystal display (LCD), light-emitting diode display (LED), plasma display panel (PDP) or the like. The user input interfaces may be wired or wireless, and may be configured to receive information from a user into the apparatus, such as for processing, storage and/or display. Suitable examples of user input interfaces include a microphone, image or video capture device, keyboard or keypad, joystick, touch-sensitive surface (separate from or integrated into a touchscreen), biometric sensor or the like. The user interfaces may further include one or more interfaces for communicating with peripherals such as printers, scanners or the like.

As indicated above, program code instructions may be stored in memory, and executed by processing circuitry that is thereby programmed, to implement functions of the systems, subsystems, tools and their respective elements described herein. As will be appreciated, any suitable program code instructions may be loaded onto a computer or other programmable apparatus from a computer-readable storage medium to produce a particular machine, such that the particular machine becomes a means for implementing the functions specified herein. These program code instructions may also be stored in a computer-readable storage medium that can direct a computer, a processing circuitry or other programmable apparatus to function in a particular manner to thereby generate a particular machine or particular article of manufacture. The instructions stored in the computer-readable storage medium may produce an article of manufacture, where the article of manufacture becomes a means for implementing functions described herein. The program code instructions may be retrieved from a computer-readable storage medium and loaded into a computer, processing circuitry or other programmable apparatus to configure the computer, processing circuitry or other programmable apparatus to execute operations to be performed on or by the computer, processing circuitry or other programmable apparatus.

Retrieval, loading and execution of the program code instructions may be performed sequentially such that one instruction is retrieved, loaded and executed at a time. In some example implementations, retrieval, loading and/or execution may be performed in parallel such that multiple instructions are retrieved, loaded, and/or executed together. Execution of the program code instructions may produce a computer-implemented process such that the instructions executed by the computer, processing circuitry or other programmable apparatus provide operations for implementing functions described herein.

Execution of instructions by a processing circuitry, or storage of instructions in a computer-readable storage medium, supports combinations of operations for performing the specified functions. In this manner, an apparatus 700 may include a processing circuitry 702 and a computer-readable storage medium or memory 704 coupled to the processing circuitry, where the processing circuitry is configured to execute computer-readable program code 706 stored in the memory. It will also be understood that one or more functions, and combinations of functions, may be implemented by special purpose hardware-based computer systems and/or processing circuitry which perform the specified functions, or combinations of special purpose hardware and program code instructions.

Many modifications and other implementations of the disclosure set forth herein will come to mind to one skilled in the art to which the disclosure pertains having the benefit of the teachings presented in the foregoing description and the associated figures. Therefore, it is to be understood that the disclosure is not to be limited to the specific implementations disclosed and that modifications and other implementations are intended to be included within the scope of the appended claims. Moreover, although the foregoing description and the associated figures describe example implementations in the context of certain example combinations of elements and/or functions, it should be appreciated that different combinations of elements and/or functions may be provided by alternative implementations without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. An apparatus for detecting a result of a test stick diagnostic device, the apparatus comprising:
a memory configured to store computer-readable program code; and
processing circuitry configured to access the memory, and execute the computer-readable program code to cause the apparatus to at least:
receive a digital image comprising a depiction of at least a portion of a test stick diagnostic device;
apply the digital image to a classifier configured to:
determine a relative position, relative orientation, and relative scale of the portion of the test stick diagnostic device with respect to the digital image;
identify a test result region of the test stick diagnostic device; and
detect a test result marking in the test result region of the test stick diagnostic device; and
provide an indication of a result of a diagnostic test based on the test result marking.

2. The apparatus of claim 1, wherein the classifier being configured to determine the relative position, relative orientation, and relative scale of the portion of the test stick diagnostic device with respect to the digital image comprises the classifier being configured to detect a position, orientation, and scale of a set of markings on a surface of the test stick diagnostic device.

3. The apparatus of claim 1, wherein the test result region of the test stick diagnostic device is an elliptical region on a surface of the test stick diagnostic device.

4. The apparatus of claim 1, wherein the test result marking is a line disposed at a predetermined location within the test result region of the test stick diagnostic device.

5. The apparatus of claim 1, wherein the apparatus being caused to receive the digital image comprises the apparatus being configured to capture the digital image via a camera incorporated into the apparatus.

6. The apparatus of claim 1, wherein the apparatus being caused to provide an indication of a result of a diagnostic test based on the test result marking comprises the apparatus being caused to display a diagnostic result message on a user interface of the apparatus.

7. The apparatus of claim 1, wherein the test stick diagnostic device is an immunological test stick diagnostic device configured to test a pregnancy status of a user.

8. A method of detecting a result of a test stick diagnostic device, the method comprising:
receiving a digital image comprising a depiction of at least a portion of a test stick diagnostic device;
applying the digital image to a classifier configured to:
determine a relative position, relative orientation, and relative scale of the portion of the test stick diagnostic device with respect to the digital image;
identify a test result region of the test stick diagnostic device; and
detect a test result marking in the test result region of the test stick diagnostic device; and
providing an indication of a result of a diagnostic test based on the test result marking.

9. The method of claim 8, wherein the classifier being configured to determine the relative position, relative orientation, and relative scale of the portion of the test stick diagnostic device with respect to the digital image comprises the classifier being configured to detect a position, orientation, and scale of a set of markings on a surface of the test stick diagnostic device.

10. The method of claim 8, wherein the test result region of the test stick diagnostic device is an elliptical region on a surface of the test stick diagnostic device.

11. The method of claim 8, wherein the test result marking is a line disposed at a predetermined location within the test result region of the test stick diagnostic device.

12. The method of claim 8, wherein receiving the digital image comprises capturing the digital image via a camera.

13. The method of claim 8, wherein providing an indication of a result of a diagnostic test based on the test result marking comprises displaying a diagnostic result message on a user interface.

14. The method of claim 8, wherein the test stick diagnostic device is an immunological test stick diagnostic device configured to test a pregnancy status of a user.

15. A computer-readable storage medium for detecting a result of a test stick diagnostic device, the computer-readable storage medium being non-transitory and having computer-readable program code stored therein that, in response to execution by processing circuitry, causes an apparatus to at least:
receive a digital image comprising a depiction of at least a portion of a test stick diagnostic device;
apply the digital image to a classifier configured to:
determine a relative position, relative orientation, and relative scale of the portion of the test stick diagnostic device with respect to the digital image;
identify a test result region of the test stick diagnostic device; and
detect a test result marking in the test result region of the test stick diagnostic device; and
provide an indication of a result of a diagnostic test based on the test result marking.

16. The computer-readable storage medium of claim 15, wherein the classifier being configured to determine the relative position, relative orientation, and relative scale of the portion of the test stick diagnostic device with respect to the digital image comprises the classifier being configured to detect a position, orientation, and scale of a set of markings on a surface of the test stick diagnostic device.

17. The computer-readable storage medium of claim 15, wherein the test result region of the test stick diagnostic device is an elliptical region on a surface of the test stick diagnostic device.

18. The computer-readable storage medium of claim 15, wherein the test result marking is a line disposed at a predetermined location within the test result region of the test stick diagnostic device.

19. The computer-readable storage medium of claim 15, wherein the apparatus being caused to receive the digital image comprises the apparatus being configured to capture the digital image via a camera incorporated into the apparatus.

20. The computer-readable storage medium of claim 15, wherein the apparatus being caused to provide an indication of a result of a diagnostic test based on the test result marking comprises the apparatus being caused to display a diagnostic result message on a user interface of the apparatus.

21. The computer-readable storage medium of claim 15, wherein the test stick diagnostic device is an immunological test stick diagnostic device configured to test a pregnancy status of a user.

22. A method of detecting a result of a test stick diagnostic device, the method comprising:
receiving a digital image comprising a depiction of at least a portion of a test stick diagnostic device;
providing the digital image to a classifier;
receiving, from the classifier, an indication of a test result marking; and
providing an indication of a result of a diagnostic test based on the test result marking;
wherein the classifier is configured to:
determine a relative position, relative orientation, and relative scale of the portion of the test stick diagnostic device with respect to the digital image;
identify a test result region of the test stick diagnostic device; and
detect the test result marking in the test result region of the test stick diagnostic device.

23. The method of claim 22, wherein the classifier being configured to determine the relative position, relative orientation, and relative scale of the portion of the test stick diagnostic device with respect to the digital image comprises the classifier being configured to detect a position, orientation, and scale of a set of markings on a surface of the test stick diagnostic device.

24. The method of claim 22, wherein the test result marking is a line disposed at a predetermined location within the test result region of the test stick diagnostic device.

25. The method of claim 22, wherein the test stick diagnostic device is an immunological test stick diagnostic device configured to test a pregnancy status of a user.
